(19) United States
(12) Patent Application Publication
(10) Pub. No.: US 2012/0301557 A1
(43) Pub. Date: Nov. 29, 2012

(54) METHOD OF ASSISTING WITH DIGESTIVE UPSETS USING A CONFECTION-BASED DELIVERY OF PEPPERMINT OIL AND GINGER

(75) Inventor: Dustin Garth James, St. Louis, MO (US)

(73) Assignee: ENTERAL HEALTH AND NUTRITION, LLC, Chersterfield, MO (US)

(21) Appl. No.: 13/328,114

(22) Filed: Dec. 16, 2011

Related U.S. Application Data
(60) Provisional application No. 61/490,203, filed on May 26, 2011.

(51) Int. Cl. A61K 36/534; A61P 1/00; A61K 36/906

(52) U.S. Cl. 424/747; 424/756

(57) ABSTRACT

A confection for assisting with the relief of common digestive complaints is provided. The confection includes a carbohydrate base and an active agent infused into the carbohydrate base. The active agent includes at least one of ginger extract or peppermint oil.

### CROSS-REFERENCE TO RELATED APPLICATIONS

The present application claims priority to U.S. Provisional Application. Ser. No. 61/490,203 filed May 26, 2011 and having the title “METHOD OF ASSISTING WITH DIGESTIVE UPSETS USING A CONFECTION-BASED DELIVERY OF PEPPERMINT OIL AND GINGER” the subject matter of which is incorporated in its entirety herein.

### BACKGROUND OF THE INVENTION

The subject matter herein relates generally to confections, and more particularly, to confections for assisting with digestive upsets through the delivery of peppermint oil and/or ginger.

Human children and adults often take ginger extract from the rhizome of Zingiber officinale and peppermint oil from the leaves of Mentha piperita for digestive health benefits, including relief of common digestive complaints. The currently available forms of ginger extract and peppermint oil are limited by their method of delivery. Both botanicals are available as dietary supplements in the form of capsules. The absorption of active compounds from such delivery methods is an inherently slow process, as the capsule needs to first degrade and then the active compounds need to be absorbed through the stomach and small intestine. The slow absorption of active compounds from ginger extract and peppermint oil from the upper digestive tract represents a shortcoming for the immediate relief of common digestive symptoms. Alternatively, a person can take peppermint oil directly into the mouth. However, the intense flavor, burning sensation, and propensity to cause mucosal damage of the upper digestive tract limit the practicality of such an approach from both a safety and organoleptic perspective.

A need remains for releasing the active compounds from ginger extract peppermint oil rapidly into the body through oral ingestion to thereby provide swift relief of common digestive upsets including cramping, urgency, stomach upset, nausea, and bloating.

### SUMMARY OF THE INVENTION

In one embodiment, a confection for assisting with the relief of common digestive complaints is provided. The confection includes a carbohydrate base and an active agent infused into the carbohydrate base. The active agent includes at least one of ginger extract or peppermint oil.

In another embodiment, a method of assisting with the relief of common digestive complaints is provided. The method includes administering, to a mammal in need thereof, a confection containing a therapeutically effective amount of an active agent. The confection includes a carbohydrate base and an active agent is infused into the carbohydrate base. The active agent includes at least one of ginger extract or peppermint oil.

In another embodiment, a method for forming a confection for assisting with the relief of common digestive complaints is provided. The method includes providing a carbohydrate base. An active agent is infused into the carbohydrate base to form a confection mixture. The active agent includes at least one of ginger extract or peppermint oil. The confection mixture is cooled into hard confections.

### BRIEF DESCRIPTION OF THE DRAWINGS

The presently disclosed subject matter will be better understood from reading the following description of non-limiting embodiments, with reference to the attached drawings, wherein below:

FIG. 1 is a flowchart of a method for forming a confection for assisting with digestive disorders.

FIG. 2 is a graph of a visual analog scale showing the results of clinical trials using the confection described in FIG. 1.

FIG. 3 is a graph showing improvements in common digestive symptoms when using the confection described in FIG. 1.

### DETAILED DESCRIPTION OF THE DRAWINGS

The foregoing summary, as well as the following detailed description of certain embodiments will be better understood when read in conjunction with the appended drawings. As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide a confection for assisting with the relief of common digestive complaints, wherein the confection includes a carbohydrate base and an active agent infused into the carbohydrate base. The active agent may include at least one of ginger extract or peppermint oil. In one embodiment, the ginger extract may include the active compound gingerol. In another embodiment, the peppermint oil includes numerous active compounds. The active compounds may include menthol. The carbohydrate base may include at least one of sugar from a source including at least one cane syrup, brown rice syrup, or corn syrup, or a sugar-free base including isomalt. The active agent may be infused into the carbohydrate base in a range of 0.01% to 25% by weight. Alternatively, the active agent may be infused into the carbohydrate base in a range of 0.4% to 5% by weight. In another embodiment, the active agent may be infused into the carbohydrate base in an amount of at least 5% by weight.

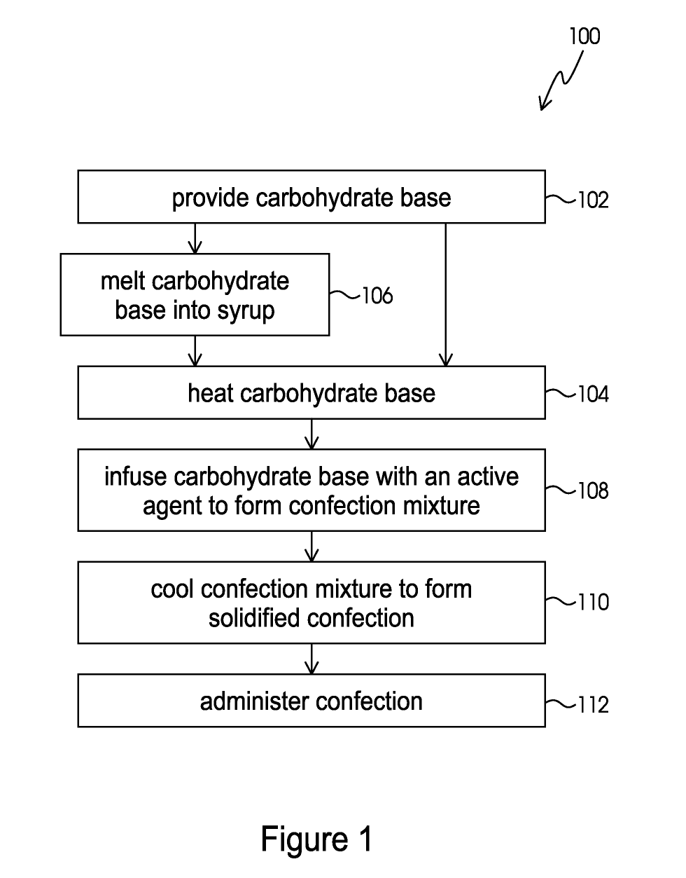

FIG. 1 is a flowchart of a method 100 for firming a confection for treating digestive disorders. The confection releases active compounds from ginger extract and/or peppermint oil rapidly into the body through oral ingestion to thereby provide swift relief of common digestive upsets including cramping, urgency, stomach upset, nausea, and bloating. At step 102, a carbohydrate base is provided. The carbohydrate base may be provided in a syrup form. Optionally, the carbohydrate base may be provided in a solid or crystallized form. In one embodiment, the carbohydrate base is a carbohydrate sugar base. For example, the carbohydrate base may include at least one of sugar from a source including at least one cane syrup, brown rice syrup, or corn syrup, or a sugar-free base including isomalt. Optionally, the carbohydrate base may be a sugar-free carbohydrate base, for example, a sugar-fee carbohydrate base including isomalt.

The carbohydrate based may be heated to an acceptable temperature for being infused with active agents, at step 104. For example, the carbohydrate base may be heated to as much as 325 degrees Fahrenheit. In an embodiment where the carbohydrate base is provided in a syrup form, the syrup may be heated directly. Alternatively, the syrup may not be required to be heated to infuse the active agent. In an embodiment where the carbohydrate base is provided in a solid or crystallized form, the carbohydrate base may first be melted, at step 106, to form syrup or a heated liquid. The syrup or heated liquid may then be heated for infusion, at step 104.

At step 108, the carbohydrate base is infused with an active agent. In one embodiment, the active agent is ginger extract. Optionally, the active agent may be peppermint oil. In another embodiment, the carbohydrate base may be infused with both ginger extract and peppermint oil. The active agent is infused into the carbohydrate base in a concentration that allows for an acceptable organoleptic profile and efficacy for relief of above stated digestive symptoms. The concentration of the active agent may range from about 0.01% to 25% by weight. In one embodiment, the concentration range is within 0.4% to 5% by weight. In one embodiment, the concentration of the active agent may be at least 5% by weight.

The carbohydrate base is infused with the active agent after the heat is removed from the carbohydrate base, but while the temperature is still high enough for mixing the carbohydrate base and the active agent. The carbohydrate base and the active agent are mixed to form a substantially uniform confection mixture. At step 110, the confection mixture is cooled to form solidified confections. The confections may be molded or shaped into drops or candies. The confection may be a confection hard candy base that may be manufactured as per confectionary industry standard protocols. When ingested, the confection is configured to dissolve into saliva through normal human physiologic prehension and mastication processes. The confection maintains its organoleptic perspective when ingested.

in one embodiment, the peppermint oil or ginger extract may be added to a liquid, such as water to make a drink. The peppermint oil and ginger extract may be added in the same concentrations as the confection. Flavorings such as carbohydrates could also be added to the liquid. The liquid may be administered alone as a drink. Alternatively, the liquid may be added to other suitable liquids to form the drink. In such an embodiment, the liquid is only cooled to room temperature. Optionally, the liquid may be refrigerated and served at a chilled temperature.

The confections containing ginger extract may contain active compounds, including gingerol. The gingerol may assist with nausea and motion sickness when ingested. The ginger extract may have at least a partial effect as an antagonist on 5HT-3 receptors in the brain that are implicated in nausea. When a person allows the confections to dissolve in their mouth, the active ingredients may enter the bloodstream, and exert at least part of their mechanism on 5HT-3 receptors to provide relief for stomach upset and nausea. The ginger extract may also have a partial effect on the mucosal lining of the digestive tract where it may reduce inflammation that can diminish common digestive complaints as well.

The confections containing peppermint oil may contain active substances, including menthol that, as part of their mechanism, may act via calcium channels to cause muscle relaxation. The digestive symptoms of cramps, urgency, and bloating often share a common pathogenesis of aberrant digestive motility as an etiology. The immediate, release of the active compounds from the peppermint oil allows for immediate relief of these complaints through direct relaxation effects on the muscles of the digestive tract. The peppermint oil also may act on pain receptors in the colon to diminish pain associated with certain foods such as mustard and pepper.

At step 112, the confection is administered. According to a method of administering the confection, when a person experiences digestive upsets, they may place the confection containing ginger extract and/or peppermint oil into their mouth. The normal human digestive processes of the mouth, including mastication and mixing of the confection with saliva, may dissolve the confection and with it, the active compounds. The blood flow through the upper digestive tract including, but not limited to, the sublingual, gastric, and proximal small bowel vasculature, may then provide for the rapid absorption of the active ingredients from the ginger extract and/or peppermint oil to enter the body and act at specific receptors that are implicit to the digestive complaints. This process allows for the rapid release of the active compounds from ginger extract and/or peppermint oil into the body to provide immediate relief of the digestive complaints.

A method of treating, digestive disorders includes administering, to a mammal in need thereof, a confection containing a therapeutically effective amount of an active agent, wherein the confection includes a carbohydrate base selected from at least one of sugar from sources that may include cane syrup, brown rice syrup, or corn syrup, or a sugar-free base including isomalt; and an active agent infused into the carbohydrate base, wherein the active agent includes at least one of ginger extract or peppermint oil. The confection may contain a therapeutically effective amount of ginger extract including the active compound gingerol. Alternatively, the confection may contain a therapeutically effective amount of peppermint oil including the active compound menthol. In one embodiment, the method includes administering a confection containing a therapeutically effective amount active of agent in a range of 0.01% to 25% by weight. In another embodiment, the method includes administering a confection containing a therapeutically effective amount active agent in a range of 0.4% to 5% by weight. In yet another embodiment, the method includes administering a confection containing a therapeutically effective amount active agent in a range of at least 5% by weight.

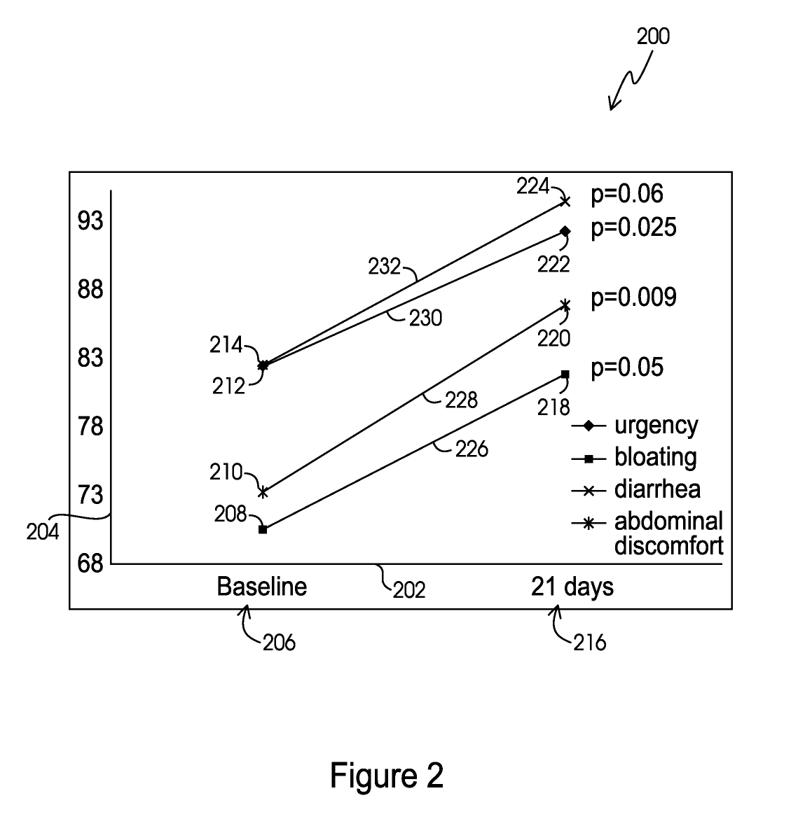

FIG. 2 is a visual analog graph 200 showing the results of clinical trials using the confection described herein. The x-axis 202 illustrates time in days. The y-axis 204 illustrates a pain scale front 0 to 100, wherein 0 represents the worst pain and 100 represents little or no pain. Fourteen people who regularly experienced gastrointestinal symptoms were surveyed at least once per week regarding their digestive symptoms. The gastrointestinal symptoms surveyed included urgency, bloating, diarrhea, and abdominal discomfort.

A baseline 206 score was taken on day 0. At baseline, the average survey result 208 for bloating was approximately 70 on the pain scale. The average survey result 210 for abdominal discomfort was approximately 74. The average survey result 212 for urgency was approximately 82. Further, the average survey result 214 for diarrhea was approximately 82.

A final survey 216 was taken after 21 days. At the final survey, the average survey result 218 for bloating was approximately 80 on the pain scale. The average survey result 220 for abdominal discomfort was approximately 85. The average survey result 222 for urgency was approximately 90. Further, the average survey result 224 for diarrhea was approximately 93.

Accordingly, on average each patient experienced improvements in their gastrointestinal symptoms. For example, the line 226 representing changes in bloating has a p value of 0.05. Additionally, the line 228 representing changes in abdominal discomfort has a p value of 0.009. The line 330 representing changes in urgency has a p value of 0.025, and the line 332 representing changes in diarrhea has a p value of 0.06. According to clinical standards, the p values show statistical significances for urgency, bloating, diarrhea, and abdominal discomfort.

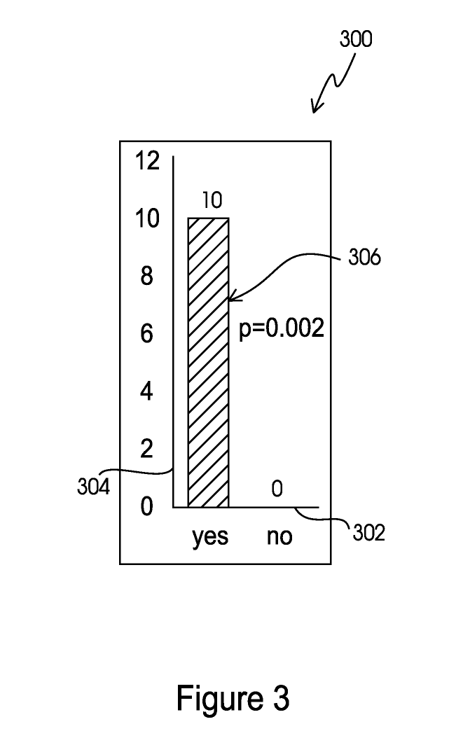

FIG. 3 is a graph 300 showing improvements in common digestive symptoms when using the confection described herein. The x-axis 302 represents the results of a yes/no survey study. The y-axis 304 represents the number of participants in the study. Each participant took at least 6 confections per day, 3 ginger and 3 peppermint. Of the 14 participants, 10 took the confections at the onset of digestive symptoms including abdominal discomfort, urgency, bloating, diarrhea, and nausea; the other 4 participants did not. Of the 10 participants who took the confections at the onset of digestive symptoms, each participant reported that the confections helped alleviate the symptoms for which they took them within five minutes, as illustrated by the bar 306. Accordingly, the test showed a p value of 0.002 which is considered strongly statistically significant.

Exemplary embodiments of a confection, a method of forming a confection, and a method of administering a confection are described above in detail, The ingredients and method steps illustrated are not limited to the specific embodiments described herein, but rather, the ingredients and method steps may be utilized independently and separately from other components described herein.

For example, the exact composition of the confection hard candy base may be modified. Moreover, the method could be modified by adjusting the ingredients to provide for more rapid dissolution of the confection base, thereby enabling more rapid release of the active compounds into the body.

Furthermore, different botanicals with distinct anticipated health benefits could be used in the confection base in addition to ginger and peppermint oil. Different products that utilize the same confection-based delivery system, but employing distinct botanicals, may also be produced.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A confection for assisting with the relief of common digestive complaints, the confection comprising:
a carbohydrate base; and
an active agent infused into the carbohydrate base, wherein the active agent includes at least one of ginger extract or peppermint oil.

2. The confection of claim 1, wherein the ginger extract includes active compounds, including gingerol.

3. The confection of claim 1, wherein the peppermint oil includes active compounds, including menthol.

4. The confection of claim 1, wherein the carbohydrate base includes at least one of sugar from a source including at least one of cane syrup, brown, rice syrup, or corn syrup, or a sugar-free base.

5. The confection of claim 1, wherein the active agent is infused into the carbohydrate base in a range of 0.01% to 25% by weight.

6. The confection of claim 1, wherein the active agent is infused into the carbohydrate base in a range of 0.4% to 5% by weight.

7. The confection of claim 1, wherein the active agent is infused into the carbohydrate base in an amount of at least 5% by weight.

8. A method of assisting with the relief of common digestive complaints comprising administering, to a mammal in need thereof, a confection containing a therapeutically effective amount of an active agent, the confection comprising:
a carbohydrate base; and
an active agent infused into the carbohydrate base, wherein the active agent includes at least one of ginger extract or peppermint oil.

9. The method of claim 8 further comprising administering a confection containing a therapeutically effective amount of ginger extract including active compounds, including gingerol.

10. The method of claim 8 further comprising administering a confection containing a therapeutically effective amount of peppermint oil including active compounds, including menthol.

11. The method of claim 8 further comprising administering a confection containing a therapeutically effective amount of active agent in a range of 0.01% to 25% by weight.

12. The method of claim 8 further comprising administering a confection containing a therapeutically effective amount active agent in a range of 0.4% to 5% by weight.

13. The method of claim 8 further comprising administering a confection containing a therapeutically effective amount active agent in a range of at least 5% weight.

14. A method for forming a confection for assisting with the relief of common digestive complaints, the method comprising:
providing a carbohydrate base;
infusing an active agent into the carbohydrate base to form a confection mixture, wherein the active agent includes at least one of ginger extract or peppermint oil; and
cooling the confection mixture into hard confections.

15. The method of claim 14 further comprising infusing the carbohydrate base with ginger extract including the active compound gingerol.

16. The method of claim 14 further comprising infusing the carbohydrate base with peppermint oil including the active compound menthol.

17. The method of claim 14 further comprising providing a carbohydrate base includes at least one of sugar from a source including at least one cane syrup, brown rice syrup, or corn syrup, or a sugar-free base.

18. The method of claim 14 further comprising infusing the carbohydrate base with the active agent in a range of 0.01% to 25% by weight.

19. The method of claim 14 further comprising infusing the carbohydrate base with the active agent in a range of 0.4% to 5% weight.

20. The method of claim 14 further comprising infusing the carbohydrate base with the active agent in an amount of at least 5% by weight.
